(19) United States
(12) Patent Application Publication
(10) Pub. No.: US 2019/0054127 A1
(43) Pub. Date: Feb. 21, 2019

(54) ANTIVIRAL AGENT AND ANTIVIRAL FOOD

(71) Applicants: EUGLENA CO., LTD., Tokyo (JP); EDUCATIONAL CORPORATION MUKOGAWA GAKUIN, Nishinomiya-shi, Hyogo (JP)

(72) Inventors: Ayaka NAKASHIMA, Kanagawa (JP); Kengo SUZUKI, Kanagawa (JP); Yuji ISEGAWA, Hyogo (JP)

(73) Assignees: EUGLENA CO., LTD., Tokyo (JP); EDUCATIONAL CORPORATION MUKOGAWA GAKUIN, Nishinomiya-shi, Hyogo (JP)

(21) Appl. No.: 16/079,609

(22) PCT Filed: Nov. 22, 2016
(86) PCT No.: PCT/JP2016/084571

§ 371 Date: Aug. 24, 2018

(30) Foreign Application Priority Data
Mar. 4, 2016 (JP) 2016-041911

(51) Int. Cl. A61K 35/68; A61K 31/716; A61P 1/04; A61P 31/14; A23L 33/10

(52) U.S. Cl. CPC A61K 35/68; A61K 31/716; A23L 33/127; A61P 31/14; A61P 1/04

(57) ABSTRACT

Provided are an antiviral agent and an antiviral food as novel methods for utilizing a Euglena-derived material. The antiviral agent and antiviral food, both comprising a Euglena-derived material as an active ingredient, are to be used for preventing or treating an infectious disease caused by an envelope-free RNA virus. Examples of the envelope-free RNA virus include rotaviruses belonging to Reoviridae. Examples of the Euglena-derived material include Euglena, a hot water extract of Euglena, paramylon and an alkali-treated paramylon.

### TECHNICAL FIELD

The present invention relates to a novel antiviral agent and a novel antiviral food, in particular, to an antiviral agent and an antiviral food both containing a Euglena-derived material as an active ingredient, for use in prevention or treatment of a viral infection.

### BACKGROUND ART

Viral infections are infectious diseases caused by invasion of viruses which are pathogens present in an environment (air, water, soil, animal, etc.), into a human body, which sometimes end up in a local epidemic, and sometimes spread in a worldwide scale due to movement of animals (especially humans), to develop into a public health problem.

Virus is a microscopic parasite generally with a size of about 0.02 μm to 0.3 μm, mainly composed of a protein shell (capsid) and a nucleic acid (RNA or DNA) inside the shell.

Completely depending on a cell for its replication, a virus first adsorbs to a host cell to invade into the cell, and then, replicates itself by releasing DNA or RNA (uncoating) within the cell, which is a process needing a specific enzyme. A host cell which is infected with a virus is disabled from functioning properly, and usually dies, and from the host cell, a new virus is released to further infect another host cell.

Viruses are roughly divided into DNA viruses having DNA, and RNA viruses having RNA, as a genome. Among the RNA viruses, rotavirus and norovirus that cause digestive organ diseases are known as typical viruses, in addition to influenza viruses that cause respiratory diseases.

Rotavirus is a virus which causes infectious gastroenteritis that is a gastrointestinal disease, which is generally known as a cause of infant diarrhea or vomiting diarrhea. In particular, infant diarrhea seen in winter is a severe diarrheal disease which causes fever, vomiting, diarrhea or dehydration symptom mainly in infants of age 2 or younger.

It is estimated that, in our country, the number of patients with rotavirus gastroenteritis reaches about 800,000, and the number of inpatients with rotavirus gastroenteritis reaches about 70,000 to 80,000 annually, with several number of deaths reported every year. Rotavirus is highly infectious, and it is supposed that nearly 100% of humans are infected with rotavirus at least once by age 5 in general, even in an advanced country with well-prepared sanitation environments. It is said that, in the United States, more than 500,000 people annually have medical consultation mainly for diarrhea symptoms, in particular, children are prone to have severe diarrhea, and about 10% of affected patients are hospitalized. Although there should be a regional difference, it is supposed that approximately around 700,000 people worldwide are killed every year (see the Non-Patent Literature 1).

An epidemiological survey in an advanced country notices that it is impossible to reduce prevalence of rotavirus by improvement of sanitary condition. Although vaccines against rotavirus of sorts have been developed, there are types or recombinants against which vaccines are ineffective, requiring countermeasures. Thus, development of a rotavirus treatment agent with a novel mechanism is being expected.

Norovirus is a virus which causes infectious gastroenteritis that is a digestive organ disease, may be a cause of a food poisoning occurred by ingestion of shellfish such as oysters, and orally infectious via feces or vomited matter of an infected human, or dust therefrom produced when dried. An outbreak of genus norovirus occurs sporadically in schools, infant facilities, elderly facilities, or the like in various parts of the world, where a dehydration symptom sometimes develops into a serious condition to cause a death.

Norovirus infection has been on an increasing trend in recent years. In some cases, norovirus repeats mutation so as to infect human to human, and lack of antibody against a new type of norovirus often causes a massive outbreak. Nevertheless, vaccines against norovirus are still under development, though some of them show some effectiveness, and thus, development of norovirus vaccine and development of norovirus treatment agent with a novel mechanism are desired.

On the other hand, Euglena (generic name: Euglena, Japanese name: Midorimushi) is attracting attention as a biological resource highly expected for use as food, feed, fuel and the like.

Euglena is equipped with as much as 59 kinds of nutrients that correspond to most of the nutrients necessary for human beings to live, such as vitamins, minerals, amino acids, unsaturated fatty acids, etc., and potential use of Euglena, such as a use as a supplement for balanced ingestion of various kinds of nutrients, or a use as a food supply source in poor areas where it is impossible to ingest necessary nutrients, has been suggested.

Mass culture of Euglena has been considered difficult for reasons such that Euglena is preyed on by predators for being located at the very bottom of the food chain, or that conditions for a culture such as light, temperature condition, stirring speed, etc. are difficult as compared to that for other microorganisms. However, in recent years, extensive researches of the present inventors have established a technique of mass culture, opening a prospect for mass supply of Euglena and paramylon extracted from Euglena.

Euglena is a unique organism which makes a flagellar movement that is an animal nature, and at the same time, has a chloroplast and photosynthesizes as a plant, and many functional characteristics are expected in Euglena itself and Euglena-derived materials.

For that reason, it is desired to elucidate functions and mechanisms through which the functional characteristics appear of Euglena and Euglena-derived materials such as paramylon which have become capable of being mass-supplied, and to develop utilization methods of these materials.

For example, the Patent Literature 1 mentions an antiviral agent containing a Euglena-derived material as an active ingredient, for use in prevention or treatment of an influenza virus infection.

Specifically, a survival rate after influenza virus infection of mice which have orally ingested Euglena or paramylon was significantly high as compared with control mice, revealing that virus titer was decreased.

In addition, the Patent Literature 2 mentions a therapeutic agent against retroviral infection containing sulfated paramylon obtained by sulfating paramylon derived from Euglena as an active ingredient.

Antiviral activities of such Euglena-derived materials have been still only partially revealed, and it is desired that functions other than the above-mentioned functions be elucidated, and methods of utilizing these materials be developed.

### CITATION LIST

### Patent Literature

PATENT LITERATURE 1: WO 2015/156339 A
PATENT LITERATURE 2: JP H04-54125 A

### Non Patent Literature

“Viruses” by Satoshi KOMOTO, Volume 64, Book 2, 2014, p. 179 to 190

### SUMMARY OF INVENTION

### Technical Problem

The present invention has been made in view of the above-described problem, and an object of the present invention is to provide a novel antiviral agent and a novel antiviral food.

Another object of the present invention is to provide an antiviral agent and an antiviral food which should be a novel utilization method of a Euglena-derived material.

### Solution to Problem

As a result of intensive research, the present inventors have found that a Euglena-derived material has an action of inhibiting growth of envelope-free RNA viruses, in particular, rotavirus and norovirus.

Particularly, while these viruses grow by adsorbing to a host cell in a living body to invade into the cell, and releasing RNA (uncoating) within the cell to replicate itself, and then releasing the replicate viruses from the host cell, the present inventors have revealed that, in the growth mechanism of these viruses, a Euglena-derived material inhibits a virus from the adsorption to a host cell, and from the replication and release within the host cell, to achieve the present invention.

The present inventors have also revealed that a Euglena-derived material inhibits an activity of a binding protein or a specific enzyme which are required in the adsorption period and the replication period of rotavirus and norovirus, in the growth mechanisms of these viruses, to achieve the present invention.

According to the antiviral agent of the present invention, the problem is solved by including a Euglena-derived material as an active ingredient, and by using the same in prevention or treatment of an infectious disease caused by an envelope-free RNA virus.

In this case, it is preferred that the envelope-free RNA virus be a virus belonging to Reoviridae, and among viruses belonging to Reoviridae, rotavirus be more preferred.

According to the structure, when a Euglena-derived material is administered, for example, to a human, especially to a virus-infected patient, the Euglena-derived material acts to inhibit a virus growth inside a living body, and thus, the present invention is capable of being used as a prophylactic agent or a therapeutic agent against a viral infection.

Further, the present invention is capable of being suitably used as a prophylactic agent or a therapeutic agent against a rotavirus infection or a norovirus infection which gives an enormous damage to a society due to its intense propagation power, among RNA virus infections.

In this case, the Euglena-derived material is preferably a hot water extract of Euglena.

Alternatively, the Euglena-derived material is preferably an alkali-treated material obtained by subjecting paramylon to an alkali treatment.

The effect of the inhibitory action against RNA virus growth is further improved by optimizing the extract of Euglena-derived material or a concentration of the extract, as described above.

Those preferred in this case are Euglena-derived, which are preferably used as a virus adsorption inhibitor for inhibiting adsorption of a virus to a cell.

According to the structure, while viruses generally grow by adsorbing to a host cell in a living body to invade into the cell, and releasing RNA (uncoating) within the cell to replicate itself, and then releasing the replicate viruses from the host cell, the extract of the present invention is capable of exerting an antiviral activity in the period of adsorption to the host cell, to inhibit the virus growth.

Therefore, it is possible, for example, to administer the present antiviral agent to a patient of viral infection at optimal administration timing, by knowing, to what period, the virus growth has progressed.

It is also possible to provide an antiviral agent for use in prevention or treatment of an infectious gastroenteritis which involves a virus as a pathogen, or an antiviral food for use in prevention or improvement of an infectious disease caused by an envelope-free RNA virus, including a Euglena-derived material as an active ingredient.

### Effects of Invention

According to the present invention, it is possible to provide a novel antiviral agent and a novel antiviral food.

In addition, it is possible to provide an antiviral agent and an antiviral food which should be a novel utilization method of a Euglena-derived material.

### BRIEF DESCRIPTION OF DRAWINGS

FIG. 1 provides data showing the result of rates of inhibiting rotavirus growth by Euglena.

FIG. 2 provides data showing the result of rates of inhibiting rotavirus growth by a hot water extract of Euglena.

FIG. 3 provides data showing the result of rates of inhibiting rotavirus growth by paramylon.

FIG. 4 provides data showing the result of rates of inhibiting rotavirus growth by amorphous paramylon.

### DESCRIPTION OF EMBODIMENTS

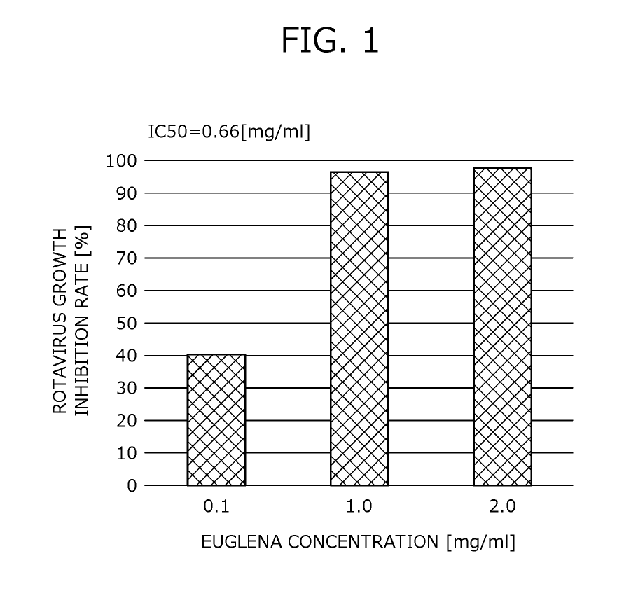

Hereinbelow, an embodiment of the present invention will be described with referring to FIG. 1 to FIG. 4.

The present embodiment relates to an invention of an antiviral agent characterized in being used in prevention or treatment of a viral infection, having a Euglena-derived material as an active ingredient, which is administered to a human to inhibit growth of a virus inside the human body.

Particularly, the present embodiment relates to an invention of an antiviral agent characterized in exerting an antiviral activity in a period of adsorption of virus to a host cell, and in a period of replication or release, in order to inhibit a virus growth.

<Outline of Viruses>

Viruses are roughly divided into DNA viruses and RNA viruses, depending on whether the genome is DNA or RNA.

DNA viruses can be classified mainly into two, depending on whether the DNA is single-stranded or double-stranded.

Specifically, there are viruses such as Parvoviridae and the like as the single-stranded DNA viruses (without an envelope), and Herpesviridae, Poxviridae, Hepadnaviridae, and the like as the double-stranded DNA viruses with an envelope, and Adenoviridae, Papillomaviridae, and the like as the double-stranded DNA viruses without an envelope.

Examples of viral diseases caused by a single-stranded DNA virus may include Human Parvo B 19 (infectious erythema), and examples of viral diseases caused by a double-stranded DNA viruses may include herpes simplex (gingivostomatitis, herpes labialis, and genital herpes virus infections), varicella zoster infections, smallpox, hepatitis B, adenovirus infections (pharyngoconjunctival fever, acute hemorrhagic conjunctivitis, epidemic keratoconjunctivitis), human papillomavirus infections.

RNA viruses can be classified mainly into three, depending on whether the RNA is single-stranded or double-stranded, and in the case of single-stranded RNA virus, whether the sense of the genome is plus-strand (+ strand) or minus-strand (− strand).

Specifically, as the − strand RNA viruses with a single strand (with an envelope), there are viruses such as Orthomyxoviridae, Rhabdoviridae, Paramyxoviridae, Filoviridae, Bunyaviridae, and Arenaviridae. Incidentally, influenza viruses belong to Orthomyxoviridae.

Examples of viral diseases caused by these − strand RNA viruses with a single strand may include influenza, avian influenza, rabies, measles, mumps (epidemic parotitis), RS virus infections (respiratory infection), Ebola (hemorrhagic fever), Marburg (hemorrhagic fever), Crimean-Congo hemorrhagic fever, SFTS, Lassa (hemorrhagic fever), Junin/Sabia/Guanarito/Machupo (hemorrhagic fever).

Next, in the + strand RNA viruses of single strand, there are viruses such as Flaviviridae, Coronaviridae, Togaviridae, Retroviridae, as those with an envelope, and there are viruses such Caliciviridae, Picornaviridae, as those without an envelope. Incidentally, norovirus belongs to Caliciviridae.

Examples of viral diseases triggered by these + strand RNA viruses of single strand may include dengue fever, West Nile fever, Japanese encephalitis, hepatitis C, yellow fever, SARS coronavirus infection, MERS coronavirus infection, rubella, human immunodeficiency (AIDS), Human T-lymphotropic (adult T-cell leukemia), hepatitis E, norovirus infection (infectious gastroenteritis), polio (acute poliomyelitis), hepatitis A, Coxsackie virus infection (hand-foot-and-mouth disease, helpangina), rhinovirus infection (common cold).

Finally, as a double-stranded RNA virus (without an envelope), there is Reoviridae, for example.

Incidentally, rotavirus belongs to Reoviridae.

Examples of viral diseases caused by double-stranded RNA viruses may include rotavirus infection (infectious gastroenteritis).

Rotavirus is an RNA virus which belongs to genus Rotavirus of Reoviridae family.

A rotavirus particle is formed with a double-shell particle composed of three layers of a core, an inner shell, and an outer-shell, and has RNA polymerase or cap synthesis-related enzyme inside the virus particle. The core is formed of proteins VP1, VP2 and VP3, covered with an inner shell protein VP6 to form a single-shell particle, and further covered with outer shell proteins VP4 and VP7 to form a double-shell particle, i.e., an infectious virus particle.

Rotavirus is classified into eight types, i.e. groups A to H, depending on antigenicity of the inner shell protein VP6. Rotaviruses reported to infect human are mainly in group A to group C.

A rotavirus infects intestinal epithelial cells of a small intestine of a human, and causes a change, i.e., a tissue lesion such as a disorder or lack in a microvillar sequence. This inhibits water adsorption from an intestine, resulting in an onset of diarrhea. The onset usually comes after an incubation period of about 48 hours, and causes acute gastroenteritis mainly in infants.

Main symptoms are diarrhea (not accompanied by bloody stool or mucous and bloody stool), nausea, vomiting, fever, abdominal pain. Although the symptoms usually heal in about 1 to 2 weeks, a dehydration, if developed severe, may cause a shock or an electrolyte imbalance, sometimes even a death.

Processes of growth inside a cell of RNA virus containing rotavirus, are described. The processes go through an “adsorption period” in which the virus adsorbs to a host cell, an “invasion period” in which the adsorbed virus invades into the cell, an “uncoating period” in which the virus invaded into the cell releases an RNA (is uncoated) within the cell, a “replication period” in which the uncoated RNA is replicated to produce new viruses, and a “release period” in which the replicate viruses are released from the cell.

A virus does not have ingredients necessary for synthesis of a nucleic acid or a protein, and necessarily requires a cell of a living body. Parasitizing within a cell of a living body, a virus grows by utilizing a metabolism of the cell, and synthesizes self-components by utilizing a material, a metabolic enzyme of the host cell, and a host cell ribosome for protein synthesis.

For example, while bacteria basically grow through binary fission, viruses increase in number at once, within a host cell which has been infected with a particle.

In the “adsorption period”, a binding protein (ligand) on the surface of a virus binds to a receptor on the surface of a host cell. A susceptibility to a virus depends on whether or not a host cell has a receptor for the virus.

In a case of rotavirus, a binding protein on the surface of a virus (outer shell proteins VP4 and VP7) binds to a receptor on the cell side.

In the “invasion period”, a virus is generally incorporated into an endosome in a cell, through an endocytosis of the cell. Then, through acidification in the endosome, a binding protein (ligand) on the virus surface fuses with a cell membrane of the host cell.

In a case of rotavirus, the outer shell protein VP4 needs to be cleaved into protein VP5 and protein VP8 by a protease (trypsin) derived from a host cell. It is considered that, after this cleavage, the protein VP8 is first brought into contact with a sialic acid-containing molecule (the first receptor), and then the protein VP5 and the outer shell protein VP7 is bound to an integrin (the second receptor), for an invasion into the cell, through a direct invasion or through an endocytosis.

In the “uncoating period”, the binding protein (capsid) of the virus invaded into the cell is degraded, and RNA is liberated within the host cell (uncoated). The period from the uncoating to the reconstitution of a virus particle, where the virus particle apparently disappears, is sometimes called as dark period.

In a case of rotavirus, the outer shell proteins VP4 and VP7 are removed at the time of cell invasion. The removal of the outer shell proteins VP4 and VP7 causes a rearrangement of the inner shell protein VP6 released inside the cell, and RNA transcription is initiated.

In the “replication period”, the uncoated RNA is incorporated into the nucleus of the host cell, which is replicated to produce a large amount of new RNA, and at the same time, a protein unique to the virus is synthesized in large amount through transcription of the RNA (synthesis of mRNA). At the time of RNA replication, an RNA-dependent RNA polymerase which serves as an RNA replication enzyme functions. At the time of synthesis of a protein from the mRNA, a protein synthesis system such as ribosome of the host cell functions. The replicate RNA and the synthesized protein gather in the cell to assemble new viruses (replicate).

In the “release period”, the virus is released outside the host cell by budding out with being covered with a cell membrane or a nuclear membrane of the host cell, or by a death of the host cell (see the Non-Patent Literature 1 for details).

Currently, there is no common antiviral agent effective against rotavirus, and main treatments are rehydration to prevent dehydration symptoms, and nutritional supplementation to prevent physical exhaustion.

Norovirus is an RNA virus belonging to Caliciviridae, which has not been succeeded yet in infection to a cultured cell or a laboratory animal, and human is said to be the only animal susceptible thereto.

Norovirus causes an acute gastroenteritis symptom such as vomiting, diarrhea in human, and is discharged in feces of a patient for about 3 to 7 days even after the symptom has disappeared, which thus requires a caution against a secondary infection.

It is considered that norovirus infects epithelial cells of a human jejunum, to cause atrophy and applanation, further exfoliation and detachment of cilia, resulting in diarrhea.

Incubation period is considered to be about 24 to 48 hours, and main symptoms are nausea, vomiting, and diarrhea, sometimes accompanied by abdominal pain, headache, fever, algor, myalgia, pharyngalgia, malaise, or the like. Although the symptoms recede without a need of special treatment, attention should be paid to dehydration or suffocation due to vomiting or diarrhea in infants, the elderly, and others with weakened physical strength.

Currently, there is no common antiviral agent effective against norovirus, and usually, symptomatic treatments are used. Main treatments are rehydration to prevent dehydration symptoms and nutritional supplementation to prevent physical exhaustion. It is considered difficult to identify norovirus infection only on the basis of clinical symptoms.

<Antiviral Agent>

“Euglena-derived material” serving as an active ingredient of the antiviral agent of the present invention includes not only Euglena or dry matter and hot water extract of Euglena, but also paramylon extracted from Euglena cells, paramylon powder and processed products of paramylon.

As the “Euglena cells”, it is desirable to use Euglena gracilis (E. gracilis), in particular, Euglena gracilis (E. gracilis) Z strain. It is also possible to use species such as Euglena gracilis Klebs, Euglena gracilis barbacillus, SM-ZK strain (chloroplast deficient strain) as a mutant strain of the Euglena gracilis Z strain, var. bacillaris as a variety of the Euglena gracilis Z strain, β-1,3-glucanase which are derived from gene mutant strain as a mutant strain of chloroplast of these species, Euglena intermedia, Euglena piride and other Euglenas such as Astaia longa.

Genus Euglena is widely distributed in fresh water such as ponds and swamps, and may be used after separated therefrom, and it is also possible to use any genus Euglena which has already been isolated.

Euglena according to the present invention involves all mutant strains thereof. These mutant strains further include those obtained through genetic methods such as recombination, transduction, transformation, etc.

In a culture of Euglena cells, those which may be used as a culture liquid are, for example, a culture liquid added with nutrient salts such as a nitrogen source, a phosphorus source, minerals, such as a modified Cramer-Myers medium ((NH4)2HPO4 1.0 g/L, KH2PO4 1.0 g/L, MgSO4.7H2O 0.2 g/L, CaCl2.2H2O 0.02 g/L, Fe2(SO2)3.7H2O 3 mg/L, MnCl2.4H2O 1.8 mg/L, CoSO4.7H2O 1.5 mg/L, ZnSO4.7H2O 0.4 mg/L, Na2MoO4.2H2O 0.2 mg/L, CuSO4.5H2O 0.02 g/L, thiamine hydrochloride (vitamin B1) 0.1 mg/L, cyanocobalamin (vitamin B12), (pH3.5)). Incidentally, the (NH4)2HPO4 may be converted to (NH4)2SO4 or NH3aq. Alternatively, it is also possible to use publicly known Hutner medium or Koren-Hutner medium which is prepared on the basis of the description of “Euglena-Physiology and Biochemistry” (edited by Shozaburo KITAOKA, Academy Publication Center, Inc.).

The culture liquid preferably has a pH of 2 or more, with a preferred upper limit of 6 or less, which is more preferably 4.5 or less. By having a pH on the acidic side, photosynthetic microorganisms can grow up more dominantly than other microorganisms, which allows suppressing contamination.

Culture of Euglena cells may be carried out by an open pond style which directly utilizes sunlight, or a condensing style which transfer sunlight collected by a concentrator through an optical fiber or the like, and radiates the transferred light in a culture tank so as to be utilized in photosynthesis.

Culture of Euglena cells may be carried out, for example, by using a supply batch method, and may be carried out by any of liquid culture methods such as a culture using a flask culture or a fermenter, batch culture method, semi-batch culture method (fed batch culture method), and continuous culture method (perfusion culture method).

Separation of Euglena cells is carried out, for example, by a centrifugation or a simple sedimentation of a culture liquid.

“Paramylon” refers to a porous polymeric body of about 700 glucose units polymerized through β-1,3-linkages (β-1,3-glucan), which is a storage polysaccharide contained in Euglena. Paramylon particles are flattened spheroid shaped particles which are formed of entangled helical β-1,3-glucan strands.

Paramylon is present as granules in Euglena cells of all species and varieties, and number, shape, and particle uniformity thereof are characterized depending on species.

Paramylon consists only of glucose, and an average polymerization degree of paramylon obtained from a wild-type strain of E. gracilis Z and a chloroplast-deficient strain SM-ZK is about 700 in glucose unit.

Paramylon is insoluble in water and hot water, but soluble in dilute alkali, concentrated acid, dimethyl sulfoxide, formaldehyde, and formic acid.

Average densities of paramylon in E. gracilis Z, and in E. gracilis var. Bacillaris SM-L1 are 1.53 and 1.63, respectively.

As for a particle size distribution, paramylon (manufactured by Euglena Co., Ltd.) has a median size of 1.5 to 2.5 μm, as measured on a laser diffraction/scattering-type particle size distribution measuring apparatus.

Paramylon particles are isolated from cultured Euglena cells by any suitable method, refined into a fine particle form, and usually provided as powder.

For example, it is possible to obtain paramylon particles by (1) culture of Euglena cells in any suitable medium; (2) separation of the Euglena cells from the medium; (3) isolation of paramylon from the separated Euglena cells; (4) refinement of the isolated paramylon; and optionally, (5) cooling and subsequent freeze-drying.

The isolation of paramylon is carried out, for example, by using a nonionic or anionic surfactant of a type which is predominantly biodegradable. The refinement of paramylon is carried out substantially simultaneously with the isolation.

In this connection, isolation from Euglena and refinement of paramylon are well known, as described, for example, in E. Ziegler, “Die naturlichen und kunstlichen Aromen” Heidelberg, Germany, 1982, Chapter 4.3 “Gefriertrocken”, DE 43 28 329, or in JP 2003-529538 A.

Examples of the “processed products of paramylon” may include amorphous paramylon and emulsion paramylon.

“Amorphous paramylon” refers to a material obtained by amorphizing crystalline paramylon derived from Euglena.

Amorphous paramylon has a relative crystallinity of 1% to 20% relative to that of crystalline paramylon produced from Euglena by a publicly known method.

For reference, this relative crystallinity was obtained by a method described in Japanese Patent No. 5612875.

Specifically, amorphous paramylon and paramylon are individually pulverized with a pulverizer (Ball Mill MM 400 manufactured by Retsh company) at an oscillation frequency of 20 times/sec for 5 minutes, and then scanned by use of an X-ray diffractometer (H'PertPRO manufactured by Spectris Inc.) at a tube voltage of 45 KV and a tube current of 40 mA in a range of 26 of from 50 to 30° to obtain diffraction peaks Pc and Pa of paramylon and amorphous paramylon in the vicinity of 2θ=20°.

Using the values Pc and Pa, relative crystallinity of the amorphous paramylon is calculated on the basis of: a relative crystallinity of amorphous paramylon=Pa/Pc×100(%).

Amorphous paramylon is an alkali-treated material which is prepared by treating crystalline paramylon powder with alkali, neutralizing the treated product with acid, and then subjecting the resultant to washing and moisture removal steps, followed by drying, in accordance with the method described in Japanese Patent No. 5612875.

“Emulsion Paramylon” is a material called emulsion paramylon for its processing method and physical properties similar to emulsified matter, which is a processed paramylon swollen by being combined with more than 4 times of water, obtained by performing a collision treatment in which a fluid obtained by adding water to paramylon is ejected from a pore nozzle at an ultra-high pressure, so that the fluid collides with an object to be collided.

Emulsion Paramylon can be obtained by performing a collision treatment one or more times at a nozzle pressure at the time of ejection of 245 MPa, with a publicly known physical property-modifying apparatus (for example, an apparatus described in JP 2011-88108 A or JP H06-47264 A) which ejects a slurry obtained by adding a water-soluble solvent to a solid such as a powder from a pore nozzle, at an ultra-high pressure, to collide the slurry with an object to be collided.

Emulsion Paramylon has a median diameter, as a particle size measured on a laser diffraction/scattering-type particle size distribution measuring apparatus, of 5 times or more of that of paramylon, which is 7 μm or more. Through an optical electron microscope, the particles are observed to be adhered to adjacent particles. Emulsion Paramylon is swollen by binding to water of 4 times or more of paramylon.

While a slurry obtained by mixing a raw paramylon with water is a free-flowing fluid, an emulsion paramylon has a viscosity due to paramylon dispersed among water molecules increasing degree of viscosity, and has such a tackiness that adhere to a hand when touched, and an elasticity, providing a touch like a glue.

Incidentally, the obtained processed paramylon is referred to as emulsion paramylon in this specification, for the treatment method and physical properties thereof. However, it is unknown whether or not the obtained processed paramylon has been emulsified, but is in a state swollen due to paramylon bound to water.

Processed products of paramylon further include water-soluble paramylon, sulfated paramylon and the like, and paramylon derivatives, obtained by chemically or physically treating paramylon by publicly known various methods.

<<Virus Growth Inhibitory Action>>

The antiviral agent exerts an antiviral action through an inhibitory action against virus growth, in particular, an inhibition action against RNA virus growth of a Euglena-derived material.

Specific action mechanism is as follows.

(1) The Euglena-derived material acts to inhibit adsorption of an RNA virus to a host cell, when a binding protein (ligand) on the surface of the virus binds to a receptor on the surface of the host cell, in the “adsorption period” of the growth process of the RNA virus.

Particularly, when the RNA virus is a rotavirus, a Euglena-derived material acts to inhibit an activity of a binding protein (outer shell proteins VP4 or VP7) or a specific enzyme involved in the adsorption period of the virus.

(2) The Euglena-derived material also acts to inhibit a replication of an RNA virus within a host cell, when a replicate RNA produced in the “replication period” in the growth process of the RNA virus gathers together with synthesized proteins to construct new viruses.

Particularly, when the RNA virus is a rotavirus, a Euglena-derived material acts to inhibit an activity of a binding protein or a specific enzyme involved in the replication period of the virus.

Accordingly, the Euglena-derived material which serves as a main active ingredient of the antiviral agent acts to inhibit a virus growth at least in the “adsorption period” and the “replication period” of the growth process of the virus, as an action not found in conventional antiviral agents.

Therefore, as compared with conventional antiviral agents which exert an antiviral activity only in a specific period in growth process of a virus, the present antiviral agent can exert an antiviral activity irrespectively in an adsorption period in the first part of a virus growth process, or in a replication period in the later part.

<<Application>>

The antiviral agent of the present embodiment can be used as a therapeutic agent against viral infection or as a therapeutic agent against viral disease, by being administered to a patient with a viral infection, or non-human animal suffering from a viral infection.

The antiviral agent of the present embodiment can also be used as a prophylactic agent against viral infections, or as a prophylactic agent against viral diseases, targeted to a human before suffering from a viral infection, a human in an incipient stage of viral infection, and non-human animal in the same stages.

In addition, the antiviral agent of the present embodiment can be used as a prophylactic agent or a therapeutic agent against infectious gastroenteritis which involves a virus as a pathogen.

The antiviral agent of the present embodiment is desirably administered, in particular, to a patient infected with a rotavirus or norovirus, among viruses.

In the case of rotavirus, the administration is desirably to a patient infected with group A rotavirus, which further desirably is group A rotavirus Wa strain (G1P [8]).

The antiviral agent of the present embodiment can be used as a composition such as a pharmaceutical composition or a food composition containing the antiviral agent that exerts the action and the effect against rotavirus and norovirus in particular, among viruses.

(Pharmaceutical Composition)

In the field of medicine, pharmaceutical compositions having the action of inhibiting a viral growth, namely, the action of inhibiting adsorption of a virus to a host cell, or the action of inhibiting a release of a virus from a host cell are provided, by blending a Euglena-derived material in an amount which allows the action to be effectively exerted, together with a pharmacologically acceptable carrier or additive. The pharmaceutical composition may be a pharmaceutical product and may also be a quasi-pharmaceutical product.

The pharmaceutical composition may be applied internally or externally. Accordingly, it is possible to use the pharmaceutical composition in a preparation form such as an agent for internal use, an injective agent for intravenous injection, subcutaneous injection, intradermal injection, intramuscular injection and/or intraperitoneal injection, an agent for transmucosal application, an agent for transdermal application, etc.

A dosage form of the pharmaceutical composition may be appropriately configured according to a form of application, and examples thereof may include solid preparations such as tablets, granules, capsules, powdered agents, and powders, liquid preparations such as liquid agents and suspension agents, and semi-solid agents such as ointments and gel agents.

(Food Composition)

In the field of food, it is possible to provide a food composition having the action of inhibiting a virus growth, by blending a Euglena-derived material in an amount effective to allow the action to be exerted inside a living body, into various foods, as a food ingredient.

That is, the present invention is capable of providing a food composition showing the use in virus growth inhibition, or the like, in the field of food. Examples of the food composition may include foods for specified health uses, foods with nutrient function claims, foods with function claims, foods for hospital patients, supplements, etc., in addition to foods in general. Use as a food additive is also possible.

Examples of the food composition may include seasonings, processed meat products, processed agricultural products, drinks (soft drinks, alcoholic drinks, carbonated drinks, milk-based drinks, fruit juice drinks, teas, coffees, nutritional drinks, etc.), powdered drinks (powdered juice, powdered soup, etc.), concentrated drinks, confectioneries (candies (throat candies), cookies, biscuits, chewing gums, gummies, chocolates, etc.), breads, cereals, etc. The foods for specified health uses, foods with nutrient function claims, foods with function claims, etc., may be those in a form of capsule, troche, syrup, granules, powder, or the like.

As used herein, the foods for specified health uses refer to foods containing a health functional component that gives an influence to a physiological function, or the like, which can be labelled as suitable for specific health use with a permission of the Commissioner of the Consumer Affairs Agency. In the case of the present invention, the foods for specified health uses correspond to foods for sale with showing, as specific health uses thereof, prevention and treatment of viral infection, inhibition of virus growth, prevention and treatment of infectious gastroenteritis, or the like.

The foods with nutrient function claims refer to foods utilized in supplementation of a nutritional component (vitamin or mineral), labeled with a function of a nutritional component thereof. For sale as a food with nutrient function claim, it is necessary that an amount of a nutritional component contained in a reference amount of daily intake be in a range between a predetermined upper limit value and lower limit value, and that not only a nutrient function claim, but also a warning notice be labeled.

The foods with functional claims refer to foods labeled with functionality based on a scientific ground, on responsibility of a business owner. Information concerning grounds of safety and functionality thereof is notified to the Commissioner of the Consumer Affairs Agency, before sale.

On the basis of the above, the present invention is capable of being used as antiviral food for specified health use, antiviral food with nutrient function claim, antiviral food with functional claim, containing a Euglena-derived material as an active ingredient, targeted to patients with a viral infection, or non-human animals suffering from a viral infection.

The present invention is also capable of being used as antiviral food for specified health use, antiviral food with nutrient function claim, antiviral food with functional claim, containing a Euglena-derived material as an active ingredient, targeted to living bodies, such as humans before suffering from a viral infection, humans in an incipient stage of a viral infection, and non-human animals in such stages.

<<Application and Dose>>

As for application of the antiviral agent of the present embodiment, in a case of rotavirus or norovirus for example, the antiviral agent is preferably prescribed so as to be dissolved in an intestine (not to be dissolved in a stomach), since rotavirus or norovirus easily infects inside a human intestine. For example, oral administration in a form of a capsule, a tablet, granules or a syrup is preferred.

### EXAMPLE

### Example 1

Euglena gracilis powder (manufactured by Euglena Co., Ltd.) was used as a Euglena-derived material. The Euglena gracilis powder 100 mg was dissolved with ethanol 1 ml, and filtered with a 0.45 μm sterilizing filter, to prepare a Euglena solution (100 mg/ml). The solution was used as an antiviral agent.

### Example 2

A hot water extract of Euglena as a Euglena-derived material was prepared according to the following procedure.

Euglena gracilis powder (manufactured by Euglena Co., Ltd.) was subjected to an extraction treatment with hot water under atmospheric pressure, and then filtered under reduced pressure to separate a residue, and a hot water extract liquid was obtained.

The prepared hot water extract liquid was filtered with a 0.45 μm sterilizing filter to obtain a hot water extract liquid (undiluted liquid) of Euglena. The extract liquid was used as an antiviral agent.

### Example 3

Paramylon as a Euglena-derived material was prepared according to the following procedure.

Euglena gracilis powder (manufactured by Euglena Co., Ltd.) was put in distilled water, and stirring was conducted at room temperature for 2 days. The resultant was sonicated to destroy cell membrane, and crude paramylon particles were collected by centrifugation. The collected paramylon particles were dispersed in 1% aqueous solution of sodium dodecyl sulfate, treated at 95° C. for 2 hours, and collected again by centrifugation. The collected paramylon particles were dispersed in 0.1% aqueous solution of sodium dodecyl sulfate and treated at 50° C. for 30 minutes. After lipid and protein were removed by this operation, the resultant was washed with acetone and ether, and then dried at 50° C. to obtain refined paramylon particles.

The prepared 100 mg of paramylon powder was dissolved with 1 ml of dimethyl sulfoxide (DMSO), and filtered with a 0.45 μm sterilizing filter to obtain a paramylon solution (100 mg/ml) The solution was used as an antiviral agent.

### Example 4

Amorphous paramylon (alkali-treated paramylon) as a Euglena-derived material was prepared according to the following procedure.

The paramylon powder prepared in Example 2 was added to a 1N sodium hydroxide aqueous solution at an amount of 5% (w/v) and dissolved. The resultant was stirred with a stirrer for 1 to 2 hours, and treated with alkali. Thereafter, a 1N hydrochloric acid was added dropwise to the 1N sodium hydroxide aqueous solution in which paramylon powder was dissolved, to cause neutralization. After repeating a step of discarding supernatant after centrifugation and washing a precipitate with distilled water, a precipitated gel was collected, frozen and then freeze-dried with a freeze dryer, to obtain amorphous paramylon.

The prepared amorphous paramylon powder 10 mg was dissolved with 1 ml of dimethyl sulfoxide (DMSO), and filtered with a 0.45 μm sterilizing filter, to obtain an amorphous paramylon solution (10 mg/ml). The solution was used as an antiviral agent.

### Test Example 1 Infection Inhibition Test in Growth Process of Rotavirus

A test to confirm action of inhibiting rotavirus growth was carried out by using the antiviral agents of Examples 1 to 4. MA-104 cells (rhesus monkey kidney cells) were used as host cells, rotavirus Wa strain (G1P [8]) was used as a virus, and DMEM (Dulbecco's Modified Eagle Medium) medium containing 10% FBS (Fetal Bovine Serum) was used as a liquid medium.

First, the MA-104 cells were seeded on a 24-well plate at 1.0×10⁴ cells/well individually, which were subjected to a monolayer culture at 37° C., under a condition of 5% CO₂, for 24 hours.

Then, the cultured MA-104 cells were infected with rotavirus at 0.1 moi (multiplicity of infection), and left for 1 hour at room temperature (to cause adsorption).

Thereafter, the Euglena solution of Example 1 was added to the liquid medium, so as to be contained at a predetermined concentration, and culture was performed in a CO₂ incubator for 48 hours.

Thereafter, the supernatant containing the virus released from the infected cells was collected, and the virus titer (FFU/ml) in the supernatant was measured by using focus reduction method, to calculate a virus growth inhibition rate (%). A concentration of the antiviral agent at which a cell infection was inhibited by 50% (IC50) was also calculated.

As objects for comparison, virus growth inhibition rates were similarly calculated in (1) an object added with the Euglena hot water extract liquid of Example 2 and cultured, (2) an object added with the paramylon solution of Example 3 and cultured, and (3) an object added with the amorphous paramylon solution of Example 4 and cultured.

### Results of Test Example 1

The test results were analyzed, and a graph which compares virus growth inhibition rates of the Euglena (Example 1) at each concentration (0.1 mg/ml, 1.0 mg/ml, and 2.0 mg/ml) is shown in FIG. 1.

The virus growth inhibition rates at each of the concentration were 40.4%, 96.8%, and 98.0%, respectively, and the IC50 was 0.66 mg/ml.

The virus growth inhibition rate was further increased, as the concentration of Euglena became higher.

In this connection, the “Euglena concentration 0.1 mg/ml” refers to such a concentration that 0.1 mg of Euglena is contained in 1 ml of liquid medium, in other words, refers to such a concentration that 0.1 volume % of Euglena solution (100 mg/ml) is contained in the liquid medium.

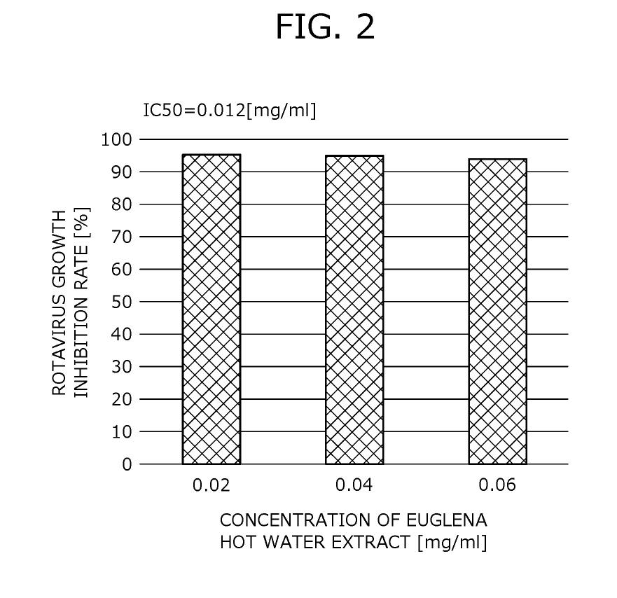

FIG. 2 shows a graph which compares virus growth inhibition rates of the Euglena hot water extract (Example 2) at each concentration (0.02 mg/ml, 0.04 mg/ml, and 0.06 mg/ml).

The virus growth inhibition rates at each of the concentration were 95.2%, 94.9%, and 93.9%, respectively, and the IC50 was 0.012 mg/ml.

The virus growth inhibition rate was 90% or more, in all of the concentrations of the Euglena hot water extract.

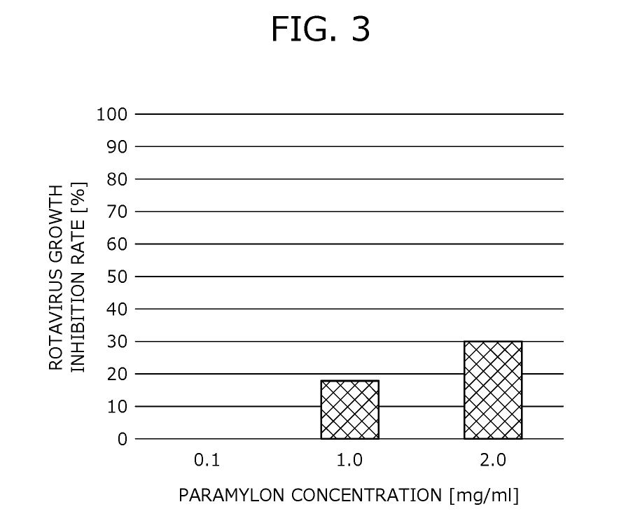

FIG. 3 shows a graph which compares virus growth inhibition rates of the paramylon (Example 3) at each concentration (0.1 mg/ml, 1.0 mg/ml, and 2.0 mg/ml).

The virus growth inhibition rates at each of the concentration were 0%, 17.8% and 30.1%, respectively.

The virus growth inhibition rate was further increased, as the concentration of paramylon became higher.

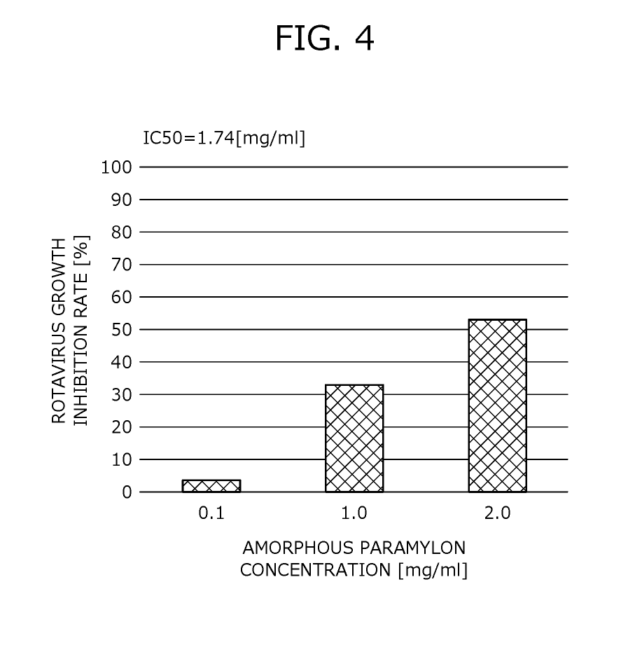

FIG. 4 shows a graph which compares virus growth inhibition rates of the amorphous paramylon (Example 4) at each concentration (0.1 mg/ml, 1.0 mg/ml, and 2.0 mg/ml).

The virus growth inhibition rates at each of the concentration were 3.8%, 33.3%, and 53.4%, respectively, and the IC50 was 1.74 mg/ml.

The virus growth inhibition rate was further increased, as the concentration of amorphous paramylon became higher.

Incidentally, these tests were performed multiple times, and high reproducibility was obtained.

### Consideration of Test Example 1

From the results of Test Example 1, action of inhibiting rotavirus growth was confirmed in those added with Euglena and those added with Euglena hot water extract at all of the concentration. In those added with Euglena, the action of inhibiting virus growth was increased in a concentration-dependent manner.

In those added with paramylon, the action of inhibiting rotavirus growth was confirmed when the concentration was relatively high (for example, 2.0 mg/ml or more). The action of inhibiting virus growth was increased in a concentration-dependent manner.

In those added with amorphous paramylon, the action of inhibiting rotavirus growth was confirmed when the concentration was relatively high (for example, 0.1 mg/ml or more). The action of inhibiting virus growth was increased in a concentration-dependent manner.

From the results of Test Example 1, it was confirmed that those added with Euglena hot water extract had the action of inhibiting rotavirus growth at lower concentration as compared with those added with Euglena.

From this, it was found that a material which is obtained more by subjecting Euglena to a hot water extract treatment had the action of inhibiting rotavirus growth.

It was also confirmed that those added with Euglena had the action of inhibiting rotavirus growth at lower concentration as compared with those added with paramylon or amorphous paramylon.

From this, it was found that among materials contained in Euglena, a material other than paramylon had the action of inhibiting rotavirus growth.

It was also confirmed that those added with amorphous paramylon (alkali-treated paramylon) had the action of inhibiting rotavirus growth at lower concentration, as compared with those added with paramylon.

From this, it was found that a material which is obtained more by subjecting paramylon to an alkali treatment had the action of inhibiting rotavirus growth.

From the result of Test Example 1, it was found that, in the action of inhibiting rotavirus growth, a suitable concentration of Euglena was 0.1 mg/ml or more, and more suitable concentration (IC50) was 0.66 mg/ml or more.

It was found that a suitable concentration (IC50) of Euglena hot water extract was 0.012 mg/ml or more.

A suitable concentration of paramylon was found to be 2.0 mg/ml or more.

It was found that a suitable concentration of amorphous paramylon was 1.0 mg/ml or more, and more suitable concentration (IC50) thereof was 1.74 mg/ml or more.

From the result of Test Example 1, it was found preferred that an antiviral agent containing Euglena as a Euglena-derived material at a concentration of at least 0.66 mg/ml or more be orally administered, in a form of a capsuled agent, a tablet, granules, a syrup, or the like, so as to be dissolved in an intestine (not to be dissolved in a stomach).

### Test Example 2 Inactivation Test of Feline Calicivirus

An inactivation test of feline calicivirus was carried out by using the antiviral agent of Example 1.

Incidentally, feline calicivirus is a virus which is widely used as an alternative virus for norovirus for which cell culture is not available.

(Cells and Media)

CRFK cells (Dai Nippon Seiyaku Kabushiki Kaisha) was used as a host cell, and feline calicivirus F9 strain (Feline calicivirus F-9 ATCC VR-782) was used as a virus.

As a cell growth medium, Eagle MEM medium “Nissui” 1 (Nissui Pharmaceutical Co., Ltd.) added with 10% FBS was used. As a cell maintenance medium, Eagle MEM medium “Nissui” 1 added with 2% FBS was used.

(Preparation of Virus Suspension)

Cell Culture

CRFK cells were monolayer-cultured in a tissue culture flask, by using the cell growth medium.

Inoculation of Virus

After the monolayer culture, the cell growth medium was removed from the flask, and feline calicivirus was inoculated. Next, the cell maintenance medium was added thereto, and culture was conducted for 1 day to 5 days, in a carbonic acid gas incubator (CO₂ concentration: 5%) of 37° C.±1° C.

Preparation of Virus Suspension

After the culture, the morphology of the cells was observed using an inverted phase difference microscope, and it was confirmed that a morphological change (cytopathic effect) has occurred in the cells. Next, the culture liquid was centrifuged (3000 rpm/min, 10 minutes), and the obtained supernatant was used as a virus suspension.

(Test Operation)

After the sample suspension of Euglena powder of Example 1 (prepared by using 99.5% ethanol) was left standing, an obtained supernatant was diluted with the cell maintenance medium, and used as a sample solution. Using the sample solution diluted by using the cell maintenance medium, a virus liquid was diluted 10-fold, and virus infectivity titer was measured. As a control, the cell maintenance medium was used, and the same test was performed.

(Measurement of Virus Infectivity Titer)

First, using the cell growth medium, CRFK cells were monolayer cultured in a tissue culture microplate (96 wells), then the cell growth medium was removed, and each 0.1 ml of the sample solution or the cell maintenance medium was added.

Next, diluent of working solution 0.1 ml was each inoculated to 4 wells, and culture was conducted for 4 to 7 days in a carbonic acid gas incubator (CO₂ concentration: 5%) of 37° C.±1° C.

After the culture, occurrence of morphological change in the cells (cytopathic effect) was observed by using an inverted phase difference microscope, and 50% tissue culture infective dose (TCID50) was calculated by Reed-Muench method, which was converted into virus infectivity titer per 1 ml of working solution.

### Results of Test Example 2

Results of Test Example 2 are shown in Table 1.

TABLE 1

| | | | log TCID₅₀/ml | | |
|---|---|---|---|---|---|
| Tested virus | Object | Concentration | Measurement-1 | Measurement-2 | Measurement-3 |
| Feline | Sample | 0.5 mg/ml | 6.5 | 6.3 | 6.5 |
| calicivirus | Control | — | 7.0 | 7.0 | 6.7 |
| | (Cell | | | | |
| | maintenance | | | | |
| | medium) | | | | |

In Table 1, “TCID₅₀” means 50% tissue culture infective dose (median tissue culture dose), and “log TCID₅₀/ml” shows common logarithm values of TCID₅₀ per 1 ml of working solution.

In this connection, at the sample concentration of 0.5 mg/ml, the cytopathic effect was not seen.

The measurement was performed three times, and evaluated as p<0.05 in a t-test.

### Consideration of Test Example 2

From the results of Test Example 2, action of inactivating feline calicivirus was confirmed in the case of adding Euglena. Feline calicivirus is a substitute virus for norovirus, and from the results of the test, it has been found that Euglena has the action of inactivating norovirus and is capable of being used as an antiviral agent against norovirus.

1-8. (canceled)

9. A method for prophylaxis or treatment of an infectious disease caused by an envelope-free RNA virus, comprising:
administering a composition comprising Euglena-derived material to a living individual in need thereof.

10. The method according to claim 9, wherein the envelope-free RNA virus is a virus belonging to Reoviridae.

11. The method according to claim 10, wherein the virus belonging to Reoviridae is a rotavirus.

12. The method according to claim 9, wherein the Euglena-derived material is a hot water extract of Euglena.

13. The method according to claim 9, wherein the Euglena-derived material is an alkali-treated material obtained by subjecting paramylon to an alkali treatment.

14. The method according to claim 9, wherein the composition is used for inhibiting adsorption of a virus to a cell.

15. A method for prophylaxis or treatment of infectious gastroenteritis which involves a virus as a pathogen, comprising:
administering a composition comprising Euglena-derived material to a living individual in need thereof.
